(19) United States
(12) Patent Application Publication
(10) Pub. No.: US 2025/0345621 A1
(43) Pub. Date: Nov. 13, 2025

(54) MEDICAL HARDWARE ENERGY DELIVERY SYSTEM

(71) Applicant: Wurth Medical Innovations, LLC, Brentwood, TN (US)

(72) Inventors: Todd R. Wurth, Brentwood, TN (US); Barry R. Hix, Birmingham, AL (US); Thomas J. Bachinski, Lakeville, MN (US)

(21) Appl. No.: 19/202,837

(22) Filed: May 8, 2025

Related U.S. Application Data
(60) Provisional application No. 63/644,868, filed on May 9, 2024.

(51) Int. Cl. A61N 2/06; A61N 1/06; A61N 1/40; A61N 2/00

(52) U.S. Cl. CPC A61N 2/06; A61N 1/06; A61N 1/40; A61N 2/002

(57) ABSTRACT

Provided herein are energy delivery systems (EDS) for use in implantable medical devices and/or as standalone endogenous energy sources, implantable medical devices comprising the EDS, and related methods of using the EDS and the implantable medical devices. The EDS is operable to generate and deliver energy (e.g., magnetic and/or electric fields) to a target area surrounding an implantation site of a patient. Such energy delivered via the EDS may accelerate tissue healing and/or maintenance and promotion of an anti-bacterial environment.

### CROSS-REFERENCE TO RELATED APPLICATION

This application claims priority to U.S. Provisional Application No. 63/644,868, filed May 9, 2024, the entire disclosure of which is hereby incorporated herein by reference.

### BACKGROUND

The field of disclosure relates generally to medical hardware such as orthopedic hardware, bone anchors, dental implants, and the like, and more particularly, energy delivery systems that can be incorporated in medical hardware.

The treatment of bone fractures has evolved with time. Plate fixation and intramedullary nails have been utilized for more than a century to fix and stabilize bone fractures. Since that time, osteosynthesis evolution has been significant with respect to advancements in surgical technique, wound management, material development, and fixation improvements. Despite these advancements, surgical hardware technology has largely remained focused on a single principle: providing fixation for tissue. Opportunities exist for further improvements and advancements in surgical hardware technology to enhance the functionality and treatment capabilities of the hardware.

### SUMMARY

Provided herein are energy delivery systems (EDS) that are operable to generate and deliver magnetic and/or electric fields to a target area proximate an implantation site. The EDS can be used as a standalone device or in conjunction with implantable medical hardware (e.g., orthopedic hardware, bone anchors, dental implants, and the like). The magnetic and/or electric fields are endogenously generated using the EDS, which can operate independent of or in conjunction with external or exogenous energy sources. The magnetic and/or electric fields provide therapy for tissue in the target area and may facilitate tissue healing, e.g., bone healing, soft tissue-to-bone healing, and soft tissue-to-soft tissue healing. The magnetic and/or electric fields may additionally or alternatively facilitate infection control in the target area, e.g., by controlling, mitigating, and/or reducing bacterial proliferation and colonization. In some embodiments, the EDS can be incorporated in implantable medical hardware to enhance and expand the existing functionality of the hardware. For example, the hardware incorporating the EDS may perform its existing function (e.g., fixing and stabilizing bone fractures) while simultaneously facilitating tissue healing, osseointegration, pain relief, and/or infection mitigation.

In one aspect, provided herein is a medical hardware device comprising a structural body implantable at an implantation site of a patient; and an energy delivery system (EDS) incorporated in the structural body, the EDS comprising componentry operable to generate, directly or passively, magnetic and/or electric fields, wherein the EDS is configured to deliver the magnetic and/or electric fields to a target area proximate the implantation site. In some aspects, the medical hardware device comprises orthopedic hardware, a bone anchor, a dental implant, a nail, a screw, a plate, an anchor, a peg, a pin, a rod, a fixator, a ring, or a bolt.

In some aspects, the componentry of the EDS comprises one or more permanent magnets. The one or more permanent magnets comprise neodymium or ceramic material. In some embodiments, the one or more permanent magnets may be moveable relative to the structural body. The one or more permanent magnets may freely float relative to the structural body such that the one or more permanent magnets are moveable in response to motion of the patient. The one or more permanent magnets may be moveable in response to an externally applied magnetic field.

In some aspects, the componentry of the EDS comprises at least two permanent magnets, wherein each adjacent pair of the at least two permanent magnets has like poles facing one another and creates a magnetic field gradient. The at least two permanent magnets may be moveable relative to one another and to the structural body, wherein movement of the at least two permanent magnets modulates the magnetic field gradient.

In some aspects, the componentry of the EDS further comprises an electrical conductor extending around a path of motion of the one or more permanent magnets, wherein movement of the one or more permanent magnets causes and/or is caused by current flowing through the electrical conductor. The electrical conductor may be electrically connected to at least one electrical contact receiving the current flowing through the electrical conductor.

In some aspects, the componentry of the EDS is positioned in a hermetically sealed housing. The housing may be sized and shaped for positioning in a hollow portion of the structural body of the medical hardware device.

In another independent aspect, provided herein is a method of treating an implantation site of a medical hardware device implanted in a patient, the method comprising: generating magnetic and/or electric fields using an energy delivery system (EDS) positioned in a subfascial, bone, or tissue region proximate the implantation site; and delivering the magnetic and/or electric fields to a target area proximate the implantation site.

In some aspects, the generating of the magnetic and/or electric fields comprises generating a constant or pulsed magnetic and/or electric field. The generating of the magnetic and/or electric fields may comprise receiving, via the EDS, magnetic and/or electric energy from an external energy source.

In some embodiments, the generating of the magnetic and/or electric fields comprises producing one or more magnetic field gradients using the EDS and modulating the magnetic field gradients. The generating of the magnetic and/or electric fields may comprise generating a flow of AC and/or DC current in a conductor of the EDS using electromagnetic interaction between the conductor and one or more permanent magnets oscillating relative to the conductor.

Objects, features, and advantages will become in part apparent and in part pointed out when reading the present disclosure in its entirety. The following description and the appended figures set forth certain features for purposes of illustration.

### BRIEF DESCRIPTION OF DRAWINGS

So that the manner where the above recited features may be understood in detail, a more particular description, briefly summarized above, may be had by reference to example aspects, some of which are illustrated in the appended drawings.

FIG. 1 is a first example of an energy delivery system (EDS);

FIG. 2 depicts the EDS of FIG. 1 incorporated in medical hardware;

FIG. 3 is a second example of an energy delivery system (EDS);

FIG. 4 depicts the EDS of FIG. 3 incorporated in medical hardware;

FIG. 5 is a third example of an energy delivery system (EDS; and

FIG. 6 depicts the EDS of FIG. 5 incorporated in medical hardware.

Corresponding reference numerals used throughout the drawings indicate corresponding elements and parts.

### DETAILED DESCRIPTION

One or more specific embodiments of the present disclosure will be described herein. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Provided herein are energy delivery systems (EDS) operable to generate and deliver magnetic and/or electric fields to a target area proximate an implantation site of a patient (e.g., a human patient). The EDS can be used as a standalone device and/or incorporated in implantable medical hardware (e.g., orthopedic hardware, bone anchors, dental implants, and the like) to equip the hardware with an endogenous energy source. The magnetic and/or electric fields generated by the EDS can provide additional functionality to the hardware, e.g., functionality beyond and in addition to using the hardware for fixing and stabilizing fractured bone or bone fragments. In some embodiments, the magnetic and/or electric fields can facilitate and/or promote healing tissue at, adjacent, or proximate the implantation site and/or controlling bacterial proliferation and colonization. In said embodiments, the EDS may reduce risk of infection by mitigating, reducing, and/or eliminating proliferation and colonization. Advantageously, this capability may operate in conjunction with and/or enhance the effects of other medical interventions and processes that contribute to infection control, thus reducing a patient's risk of infection. In this way, the implantable medical hardware incorporating the EDS hereof may positively influence the area at or proximate the implantation site including by accelerating tissue healing, promotion of osseointegration, maintenance and promotion of an anti-bacterial environment, pain relief, and/or infection mitigation.

In various embodiments, the EDS may be incorporated in medical hardware, which may be implantable in a patient. For example, the EDS may be incorporated with any medical hardware for use with orthopedic treatment and/or wound management. Nonlimiting examples of hardware include nails (e.g., intramedullary nails), bone screws, bone plates, bone anchors, bone pegs or pins, spinal fusion devices, joint arthroplasty devices, dental implants, rods, internal fixators, external fixators, rings, bolts, and the like. Such applications are provided by way of example and do not limit the applications or medical hardware that can incorporate the EDS. In some embodiments, the EDS may be incorporated in existing medical hardware to retrofit such devices with the energy delivery functionality described herein.

In some embodiments, the EDS may be installed within a hollow portion of the hardware, such as within a hollow portion or cannula of a cannulated screw. In some embodiments, the EDS may be installed within a structural body of the hardware, e.g., within a wall of a nail or screw, within a body of a plate or implant, etc. In some embodiments, the EDS may be installed on an outer surface of the hardware, e.g., on the surface of a screw, nail, plate, etc.

The EDS comprises componentry (e.g., one or more magnets and/or one or more conductors) that facilitates transfer of energy in the form of a magnetic and/or electric field. The componentry of the EDS may operate to generate magnetic and/or electric fields, directly or passively, which can be delivered to the target area proximate the implantation site to facilitate and/or promote accelerated tissue healing and infection control. In some embodiments, the componentry of the EDS facilitates well-controlled delivery of the magnetic and/or electric field to a desired or target location of the area proximate the implantation site.

The componentry may comprise magnetic components, such as permanent magnets. The magnetic components may comprise neodymium, ceramic, other suitable magnetic material, or combinations thereof. In some embodiments, the componentry may be housed in a housing (e.g., a cartridge or a tube). The housing may be hermetically sealed. The housing may be flexible or rigid depending on the intended application. The housing may enable standalone usage of the EDS and/or incorporating the EDS in medical hardware. In some embodiments, the componentry may be directly installed in and/or disposed on the medical hardware (e.g., without the use of a housing). The componentry and/or housing can have various configurations (e.g., diameters, sizes, shapes, lengths, etc.) to enable the EDS to function as described herein. The configuration of the componentry and/or housing can vary depending on the intended or desired hardware with which the EDS is used or whether the EDS is used as a standalone device. In some embodiments, the componentry, and optionally the housing, may comprise or may be incorporated in a screw, peg, pin, or other fastener or fixation means which may be used as a standalone implantable device or may be integrated with other or additional medical hardware. In some embodiments, the componentry, and optionally the housing, could be used in conjunction with (and optionally attached to) a separate medical hardware device and the componentry of the EDS may be operable to produce and/or propagate a magnetic and/or electric field to the attached or adjacent medical device.

The componentry of the EDS (e.g., magnetic components and/or electrical conductors) may be operable to produce a constant or pulsed magnetic and/or electric fields. In some embodiments, the componentry may be configured to receive energy (e.g., magnetic and/or electric energy) from an exogenous or external energy source, such as, for example and without limitation, a generator, electrical stimulator, pulsed or constant magnetic field device, external magnet, and the like. In this way, the componentry may be operable as a primary or secondary endogenous energy source in conjunction with a secondary or primary exogenous energy source. In one example, an exogenous magnetic device (e.g., a magnetic patch) may be positioned over or proximate an incision for the EDS and/or hardware incorporating the EDS, and may operate as a primary or secondary influencing magnetic field for passively directing the componentry of the EDS.

In some embodiments, magnetic componentry of the EDS may include one or more moveable magnets, e.g., “free floating” or oscillating magnets. Such moveable magnets may provide magnetic motion, which could be controlled via an exogenous magnetic influence and/or caused in response to motion of the patient. In some embodiments, the magnetic motion may provide inductive force for generating electrical energy in a conductive wire (e.g., wrapped around the housing and/or the hardware to surround the magnetic componentry). The generated electrical energy may include AC and/or DC microcurrents and pulse and/or constant currents. Additional electronic components may be included in the EDS to control the generation of electrical energy.

In some embodiments, the componentry of the EDS may cooperate with the structure of the housing and/or the medical hardware to enhance functionality in conjunction with the delivery of the magnetic and/or electric fields to the target area. Various modifications could be made to the structure of the EDS housing and/or the medical hardware for this purpose. For example, the housing of the EDS and/or the medical hardware may be selectively coated with, or otherwise selectively incorporate, electrically insulating material or magnetic shielding material to control direction and/or intensity of the magnetic and/or electric fields. In some embodiments, the housing of the EDS and/or medical hardware may include perforations or openings in an outer surface that utilize the indwelling magnetic field to facilitate bone-to-housing or bone-to-hardware ingrowth, which may facilitate accelerated and improved fixation between bone and the housing or medical hardware.

### Magnetic Fields

In some embodiments, the EDS is operable to deliver one or more magnetic fields to the target area proximate the implantation site. Magnetic fields can have beneficial effects on bone and soft tissue healing. In soft tissue, magnetic fields may significantly up-regulate gene expression and down-regulate inflammatory gene expression in tenocytes (tendon cells) resulting in improved early tendon-to-tendon and tendon-to-bone healing. Similar results may be realized in dermal tissue subjected to magnetic field therapy. In bone tissue, magnetic fields may promote osteoblast activity and inhibit the differentiation of osteoclasts. By doing so, magnetic fields can positively influence the very basis of cellular bone metabolism. This influence affords magnetic fields the ability to facilitate bone healing and maintain bone health via the prevention of osteoporosis, for example.

The EDS provided herein may also provide favorable antimicrobial effects via the delivery of magnetic fields which may alter the permeability of bacterial cell membrane ion channels, increase the formation of free radicals within bacterial cells, and aid in the disintegration of the bacterial cell wall. In doing so, magnetic fields delivered via the EDS can enable the medical hardware incorporating the EDS the ability to positively influence tissue healing while negatively influencing bacterial colonization. This functionality can significantly enhance the functionality and increase the versatility of the medical hardware incorporating the EDS and improve the ability to facilitate and influence tissue management.

Advantageously, the EDS can provide an endogenous source for delivering magnetic fields. In this way, the EDS may overcome disadvantages associated with exogenous sources of magnetic fields to influence bone and soft tissue healing. Despite the improved ability of the magnetic field to penetrate tissue, e.g., compared to electric fields and/or light wavelength therapy, the magnetic field is inherently disorganized with respect to the target area for site. The EDS provided herein may facilitate a directional magnetic gradient at the tissue repair site by harnessing the energy of an endogenous magnetic field source. In some embodiments, the EDS can provide an endogenous magnetic field source and may be utilized with an exogenous magnetic field source. For example, the EDS may harness the energy of an exogenous magnetic field source. In some embodiments, the EDS may be incorporated in medical hardware being utilized for, e.g., tissue repair, fixation, bone stability, or the like, such that any additional risk of wound or incisional infection can be reduced or eliminated.

### Electric Fields

In some embodiments, the EDS is operable to deliver one or more electric fields to the target area proximate the implantation site. The human body is full of electrical gradients. Direct electrical current is created by the biological movement of ions across cell membranes. In normal states, these electrical gradients are carried along tissue planes and bodily structures via organized pathways. When tissue is injured, the electrical gradient becomes disorganized. This disorganization adversely affects the speed at which tissues heal and the quality of tissue that is produced. Delivering electric fields or electrical gradients can positively influence and have a beneficial effect on tissue healing. Moreover, the EDS may advantageously create electrical gradients that have specific direction (polarity) and amplitude to create an environment that is more conducive to healing.

Electric fields may be utilized in the healing of cutaneous injuries and Achilles tendon injuries. Electric energy sources are typically exogenous, having a superficial location and applied directly on the skin. The EDS as described herein may advantageously generate and control delivery of the electric field in subfascial locations, or when the EDS is installed in bone or bone tissue. For example, the EDS may facilitate creating a directional electrical gradient at the tissue repair site by harnessing the energy of an exogenous source and/or creating one endogenously. In some embodiments, the EDS may be incorporated in medical hardware being utilized for, e.g., tissue repair, fixation, bone stability, or the like, such that any additional risk of wound or incisional infection can be reduced or eliminated.

The electric fields generated via the EDS may also provide favorable antimicrobial effects via their ability to activate macrophages. Macrophages function to eliminate microorganisms, remove dead cells, and stimulate other immune system cells. As such, macrophages facilitate healthy wound repair. Upregulation of macrophages in electric field therapy is desirable particularly in the early phases of tissue healing. In some embodiments, the electric field may be controlled to change from negative to positive at certain points along the tissue healing spectrum, which may facilitate preferentially upregulating the antimicrobial and healing attributes of the electric field to facilitate optimizing benefit for tissue healing.

### Example Medical Hardware Incorporating the EDS

As discussed herein, the EDS may be utilized as a standalone device or incorporated in medical hardware. When incorporated in medical hardware, the EDS facilitates enhancing the functionality of the medical hardware via the delivery of magnetic and/or electric fields to the target area proximate the implantation site of the medical hardware. In this way, the medical hardware comprising the EDS may be used to promote tissue healing, facilitate infection control, and promote an anti-bacterial environment, in addition to the conventional functionality provided by the medical hardware such as internal fixation, stability, fusion, etc. Non-limiting examples of medical hardware will now be described; however it is understood that the EDS may be incorporated in and/or used in conjunction with any suitable medical hardware without departing from the scope of this disclosure.

### Orthopedic Hardware

Advancements in modern orthopedic hardware have primarily concentrated on plate design (pre-contoured, low-profile), locking capabilities, alterations in modulus of elasticity, and ease of usage. These advancements have proven beneficial in fracture management but have not changed the role of hardware in a substantive manner. The solitary role of hardware providing tissue support and stability has remained unchanged. By incorporating an EDS as described herein for the delivery of magnetic and/or electric fields via orthopedic hardware, the orthopedic hardware may be transformed into an instrument that additionally promotes tissue healing/maintenance and assists with control of bacterial colonization/proliferation. In some embodiments, the orthopedic hardware comprising the EDS may include nails (e.g., intramedullary (IM) nails), bone screws (e.g., cannulated screws), bone anchors, bone plates, bone pegs or pins, spinal fusion devices, joint arthroplasty devices, dental implants, rods, internal fixators, external fixators, rings, bolts, and the like.

In some embodiments, an IM nail comprises an EDS as described herein. The EDS can be incorporated in a hollow or cannulated portion of the IM nail, within a wall of the IM nail, or on an outer surface of the IM nail. The EDS may enable the IM nail to facilitate fracture healing, control of, and/or reduction in, bacterial proliferation and colonization, and maintenance of surrounding bone quality after fracture healing.

In some embodiments, a bone screw comprises an EDS as described herein. The bone screw may be a cannulated screw. The EDS can be incorporated in bone screws (e.g., cannulated screws) of varying sizes to facilitate bone healing, stabilization, and/or maintenance while simultaneously controlling and/or reducing local bacterial proliferation and colonization. The EDS can be incorporated in a hollow or cannulated portion of the bone screw, within a wall of the bone screw, or on an outer surface of the bone screw.

In some embodiments, a bone plate or plate construct comprises an EDS as described herein. In some embodiments, EDS may be incorporated in the bone plate via the use of pegs, pins, screws, or the like. The EDS may be housed in and/or positioned on a peg, pin, screw, or the like of the bone plate to provide internal magnetic and/or electric field delivery capabilities. Such pegs, pins, screws, or the like of the bone plate incorporating the EDS can be positioned at, adjacent, or proximate areas in need of bone healing assistance such as fractures, osteotomies, or nonunions. In some embodiments, the EDS can be incorporated within the structure of the plate and/or on an outer surface of the plate. The EDS incorporated in the bone plate can facilitate bone healing, stabilization, and/or maintenance while simultaneously controlling and/or reducing local bacterial proliferation and colonization of the plate and surrounding tissue.

In some embodiments, an implantable peg or pin comprises an EDS as described herein. The EDS can be incorporated in a hollow or cannulated portion of the peg or pin, within a wall of the peg or pin, or on an outer surface of the peg or pin. The EDS may enable the peg or pin to be utilized in isolation to facilitate expedited tissue healing and control and/or reduction of bacterial proliferation and colonization at the implantation site or site of use. In some embodiments, the peg or pin may be utilized for the treatment of osteoporosis in sites at risk for fragility fractures.

In some embodiments, an implantable spinal fusion device (or implant) comprises an EDS as described herein. The EDS may be incorporated in the spinal fusion device to facilitate fusion healing and controlling and/or reducing bacterial proliferation and colonization of the surgical site.

In some embodiments, an implantable joint arthroplasty device (or implant) comprises an EDS as described herein. The EDS may be incorporated in the joint arthroplasty device to facilitate bone ingrowth, prolonged bone maintenance, and controlling and/or reducing bacterial proliferation and colonization of the surgical site. The EDS may be integrated within a hollow portion or structural body of the joint arthroplasty device and/or on an outer surface of the device. In some embodiments, the EDS incorporated in the joint arthroplasty device may be operable to produce magnetic and/or electric fields autonomously as an endogenous energy source. In some embodiments, the EDS incorporated in the joint arthroplasty device may be used in conjunction with an external or exogenous energy source to produce magnetic and/or electric fields.

### Bone Anchors

In some embodiments, a bone anchor comprises an EDS as described herein. Advancements in bone anchor technology have primarily relied upon changes in anchor composition, improvements in pullout strength, and alterations in suture material. Ultimately, the role of the anchor in providing stability for attachment of soft tissue to bone has remained substantially the same. Incorporating the EDS to generate and deliver magnetic and/or electric fields via the bone anchor may facilitate promoting the healing of the soft tissues at the bone interface site while simultaneously assisting with the control and/or reduction of bacterial proliferation and colonization at the site.

### Dental Implants

In some embodiments, a dental implant comprises an EDS as described herein. The use of dental implants has dramatically expanded over the last two decades. Increased incidence of tooth-related problems from unhealthy lifestyles and the growing acceptance of cosmetic dentistry are factors fueling this growth. These implants rely on bone ingrowth for long term stability and function. Common complications include peri-implant bone loss, infection, and edema. Incorporating the EDS to generate and deliver magnetic and/or electric fields via the dental implant may facilitate bone ingrowth, maintaining and stabilizing the bone tissue around the implant after ingrowth, and controlling and/or reducing bacterial proliferation and colonization in the surrounding tissue.

### Smart Device

In some embodiments, the EDS may be incorporated into medical hardware that is implanted into a patient and used in combination with an exogenous or external energy source, such as, for example and without limitation, a generator, electrical stimulator, pulsed or constant magnetic field device, external magnet, and the like. In one example, the implanted EDS is used in combination with an external pulsed electromagnetic field (PEMF). The field effect may be adjusted or customized to a patient via the influence of an external source on the implanted EDS. In such embodiments, a medical provider may adjust the field effect based on clinical information gathered during the patient's course of treatment after the medical hardware EDS has been implanted. For example, the magnetic and/or electric fields that are endogenously generated via the EDS may be used to gauge surrounding tissue via an exogenous source to estimate healing, such as tissue healing at, near, or proximate to a site of implantation, the medical hardware, the EDS, and/or the endogenous electromagnetic field. A medical provider may use the information to customize a patient's treatment by, for example, adjusting the field effect for improved tissue management and maximizing positive clinical outcomes.

### Nanomedicine Incorporation

The medical hardware incorporating the EDS may be used in combination with magnetic nanoparticles. In some embodiments, the medical hardware EDS comprises a platform for magnetic nanoparticle drug delivery. Current magnetic nanoparticle treatments utilize an external magnetic field to localize their field of influence. However, the medical hardware EDS as described herein may allow for targeted magnetic nanoparticle medical applications, particularly by use of, for example, the endogenous electromagnetic field generated via the EDS. Such embodiments may provide a significant advancement in the fields of oncology, infection, trauma, or any other clinical applications in which drug delivery system is utilized via a magnetic nanoparticle application.

### Illustrated Embodiments

Example embodiments of energy delivery systems (EDS) operable to generate and deliver magnetic and/or electric fields in accordance with the present disclosure will now be described with reference to the drawings. The EDS of the illustrated embodiments may be used as standalone devices and/or may be incorporated in the medical hardware described herein. The EDS of the illustrated embodiments may include any of the features and functionalities described herein. Additionally, the EDS of the illustrated embodiments may be incorporated in any of the medical hardware described herein. The illustrated embodiments depict non-limiting examples of medical hardware in which an EDS can be incorporated. In various embodiments, the EDS of the illustrated embodiments may be incorporated in various medical hardware as described herein, such as orthopedic hardware, bone anchors, dental implants, and the like. Elements and features described with reference to one illustrated embodiment are not limited to that embodiment only; the features and elements of the illustrated embodiments can be utilized in any of the other illustrated embodiments any combination.

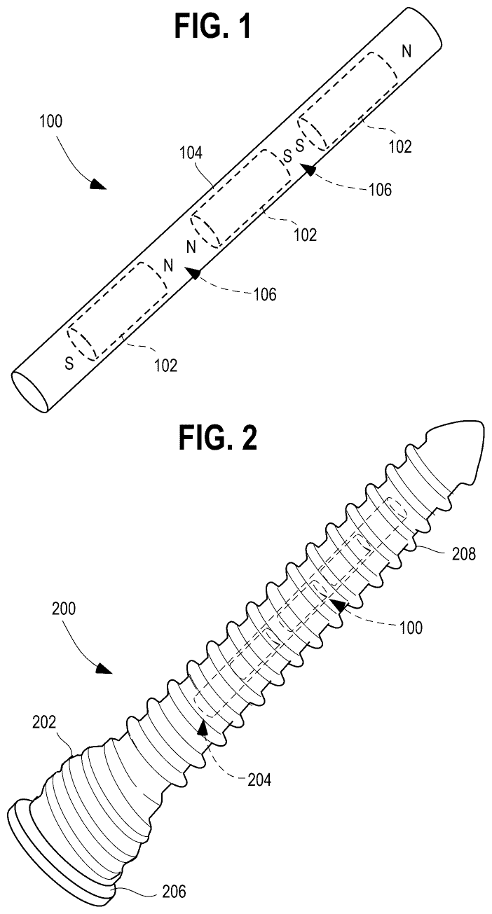

Referring now to FIG. 1, a first example of an energy delivery system (EDS) is indicated at 100. The EDS 100 is in the form of a cartridge assembly comprising one or more magnets 102 positioned in a housing 104. The housing 104 in this example is a hermetically sealed tube. The housing 104 may be of any material and any transparency (e.g., opaque, substantially opaque, semi-transparent, substantially transparent, or transparent) depending on the intended application of the EDS 100. The housing 104 may be flexible or rigid. The housing 104 has a cylindrical shape. The configuration (e.g. size and/or shape) of the housing 104 may vary. For example, the housing 104 may have various diameters, sizes, shapes, lengths, etc. depending on the intended application of the EDS 100. The EDS 100 may be used as a standalone device or may be incorporated in medical hardware (e.g., the medical hardware 200 of FIG. 2).

The magnet(s) 102 may comprise any suitable magnetic material. In some embodiments, the magnet(s) 102 comprise permanent magnet(s). For example, the magnet(s) 102 may comprise neodymium, ceramic, other suitable magnetic material, or combinations thereof. In the illustrated embodiment of FIG. 1, the EDS 100 comprises three magnets 102. In other embodiments, more or fewer magnets 102 may be included (e.g., one, two, three, four, five, or more than five magnets). The magnets 102 may have a cylindrical shape that complements a cylindrical shape of the housing 104. The configuration (e.g., size and/or shape) of the magnets 102 may vary. For example, the magnets 102 may have various diameters, sizes, shapes, lengths, etc. depending on the intended application of the EDS 100. In some embodiments, the size and/or shape of the magnet(s) 102 may vary to complement the size and/or shape of the housing 104. The magnets 102 may be similarly configured (e.g., having similar size and shape) or the magnets 102 may have dissimilar configurations (e.g., having different sizes and/or shapes).

In the illustrated example of FIG. 1, the magnets 102 are moveably positioned in the housing 104. For example, the magnets 102 may freely float or oscillate within the housing 104. Such movement may be caused by movement of the patient when the EDS 100 is implanted. Additionally or alternatively, the magnets 102 may move in the housing 104 in response to an externally applied magnetic field. Such external magnetic fields may be applied via exogenous magnetic field generators, pulsed or constant magnetic field devices, magnetic patches, or the like. In one example, an exogenous magnetic device (e.g., a magnetic patch) may be positioned over or proximate an incision for the EDS 100 and/or hardware incorporating the EDS 100, and the exogenous magnetic device may operate as an influencing magnetic field for passively causing movement of the magnets 102 within the housing 104. Movement of the magnets 102 in the housing 104 may modulate magnetic gradients 106 created between the magnets 102. For example, the magnetic gradients 106 may modulate in volumetric area and strength in response to movement of the magnets 102. In some embodiments, the magnets 102 may be fixed within the housing 104 such that the magnets 102 do not substantially move.

As shown in FIG. 1, adjacent pairs of magnets 102 may be positioned with like poles facing one another. For example, in the illustrated example, one pair of adjacent magnets 102 is positioned with the respective north (N) poles facing one another and another pair of adjacent magnets 102 is positioned with the respective south(S) poles facing one another. The like poles of the adjacent pairs of magnets 102 create the magnetic field gradients 106 between the magnets 102. In the illustrated example, two magnetic field gradients 106 are created by the two pairs of adjacent magnets 102. More or fewer magnetic field gradients 106 may exist depending on the number of magnets 102 included in the EDS 100.

The magnetic field gradients 106 provide a directional magnetic field gradient that can be delivered via the EDS 100 at an implantation site, e.g., a tissue repair site or a bone fracture. The magnetic field gradient may facilitate the benefits of magnetic fields at the implantation site as described herein, such as, for example, promoting the healing of the soft tissues, controlling bacterial proliferation and colonization, promoting bone healing, soft tissue-to-bone healing, and soft tissue-to-soft tissue healing, etc. The magnetic field gradients 106 can be induced by the inherent magnetism of the magnets 102 with adjacent pairs of the magnets 102 having like poles facing one another. In some embodiments, the magnetic field gradients 106 can be induced via the magnets 102 and an external or exogenous magnetic field source that influences the magnets 102. In some embodiments, as described above, the magnets 102 can move (e.g., float or oscillate) in the housing 104 such that the magnetic field gradients 106 are modulated (e.g., in volumetric area and strength).

FIG. 2 depicts the EDS 100 incorporated in medical hardware 200. The medical hardware 200 incorporating the EDS 100 may include any medical hardware, including but not limited to orthopedic hardware, bone anchors, dental implants, and the like. In the illustrated example, the medical hardware 200 is an orthopedic screw. The medical hardware 200 has a structural body 202 that incorporates the EDS 100. In the illustrated example, the EDS 100 is positioned in a hollow or cannulated portion 204 of the body 202. In other embodiments, the EDS 100 may be positioned within a wall of the body 202, on an outer surface of the body 202, or at another suitable location to enable the EDS 100 to function as described herein.

In the illustrated embodiment, the EDS 100 comprises the magnets 102 positioned in the housing 104 when incorporated in the medical hardware 200. In some embodiments, the EDS 100 may be provided without the housing 104 when incorporated in the medical hardware 200. For example, the magnets 102 may be freely positioned in the hollow portion 204 of the body 202, within a wall of the body 202, on the outer surface of the body 202, etc. without the housing 104.

The EDS 100 may be incorporated in the medical hardware 200 using any suitable means to install the EDS 100. In some embodiments, the EDS 100 may be positioned in the hollow portion 204 and secured therein using adhesive and/or interference fit. In some embodiments, the housing 104 of the EDS 100 may be secured within the hollow portion 204 such that the housing 104 does not substantially move relative to the hardware 200. The magnets 102 may move relative to the housing 104 and relative to the hardware 200. The EDS 100 may be installed before or after implanting the medical hardware 200 in a patient.

In the illustrated embodiment of FIG. 2, the medical hardware 200 is an orthopedic screw that comprises a head 206 and a threaded tail 208. The threaded tail 208 may be utilized to drive the screw into an implantation site (e.g., bone or tissue) via rotation. Rotation of the tail 208 may be driven via manipulating the head 206 using a suitable tool. The EDS 100 may be positioned toward or proximate the tail 208 such that the EDS 100 is positioned within the implantation site. In some embodiments, the EDS 100 may be positioned toward or proximate the head 206.

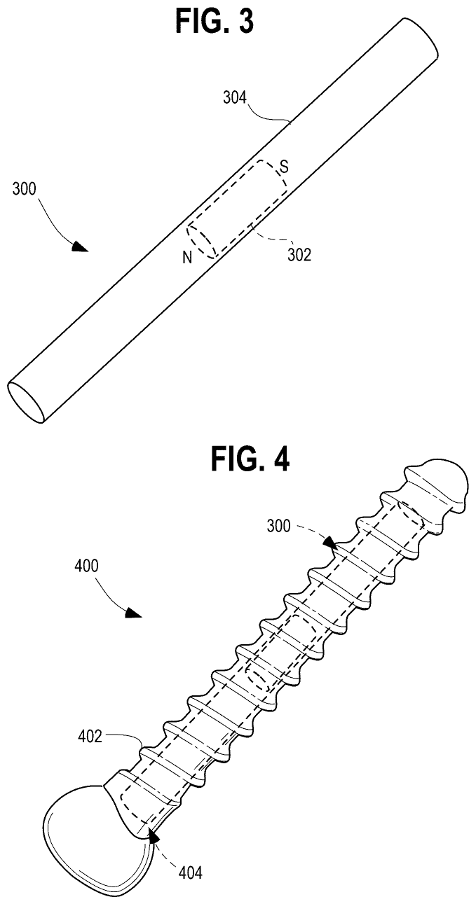

Referring now to FIG. 3, a second example of an energy delivery system (EDS) is indicated at 300. The EDS 300 may include similar components to those of the EDS 100 of FIG. 1, for example, the EDS 300 comprises a magnet 302 and a housing 304. The housing 304, like the housing 104 described above, may be a hermetically sealed tube having a cylindrical shape. Description of the housing 104 applies to the housing 304 unless expressly stated otherwise or the context clearly indicates otherwise. The EDS 300 may be used as a standalone device or may be incorporated in medical hardware (e.g., the medical hardware 400 of FIG. 4).

In this example, the EDS 300 includes the single magnet 302. The magnet 302 may be similar in construction to the magnets 102 described above. For example, the magnet 302 may comprise a permanent magnet. In some embodiments, the magnet 302 may comprise neodymium, ceramic, other suitable magnetic material, or combinations thereof. The size and shape of the magnet 302 may vary, e.g., the magnet 302 can have various diameters, sizes, shapes, lengths, etc. depending on the intended application of the EDS 300 and/or size and shape of the housing 304. In the illustrated example, the magnet 302 has a cylindrical shape that complements the cylindrical shape of the housing 304. The magnet 302 may also be moveable within the housing 304 as described above for the magnets 102, e.g., the magnet 302 may freely float or oscillate within the housing 304. Movement of the magnet 302 may be caused by movement of the patient when the EDS 300 is implanted. Additionally or alternatively, the magnet 302 may move in the housing 304 in response to an externally applied magnetic field. Such external magnetic fields may be applied via exogenous magnetic field generators, pulsed or constant magnetic field devices, magnetic patches, or the like. Movement of the magnet 302 in the housing 304 may modulate the position and/or direction of the magnetic field created by the magnet 302. For example, the magnet 302 may move in the housing 304 to produce an oscillating magnetic field when the patient is in motion. In some embodiments, the magnet 302 may be fixed within the housing 304 such that the magnet 302 does not substantially move.

The magnet 302 may operate as a primary (or single) magnetic field for the EDS 300. In some embodiments, the magnet 302 may be influenced by an externally applied magnetic field. The magnet 302 may operate as the primary magnetic field influenced by a secondary, externally applied magnetic field, or the magnet 302 may operate as the secondary magnetic field influenced by a primary, externally applied magnet field. Such external magnetic fields may be applied via exogenous magnetic field generators, pulsed or constant magnetic field devices, magnetic patches, or the like.

FIG. 4 depicts the EDS 300 incorporated in medical hardware 400. The medical hardware 400 incorporating the EDS 300 may include any medical hardware, including but not limited to orthopedic hardware, bone anchors, dental implants, and the like. In the illustrated example, the medical hardware 400 is an orthopedic screw. The medical hardware 400 of FIG. 4 could incorporate the EDS 100 of FIG. 1; the EDS 300 could also be incorporated in the medical hardware 200 of FIG. 2. The medical hardware 200, 400 may include similar components (e.g., a hollow or cannulated portion, head, threaded tail, etc.), and description of the medical hardware 200 is applicable to the medical hardware 400, and vice versa, unless expressly stated otherwise or the context clearly indicates otherwise.

As shown in FIG. 4, the EDS 300 may be incorporated in a structural body 402 of the hardware 400. In the illustrated example, the EDS 300 is positioned in a hollow or cannulated portion 404 of the body 402. In other embodiments, the EDS 300 may be positioned within a wall of the body 402, on an outer surface of the body 402, or at another suitable location to enable the EDS 300 to function as described herein. As described above for the EDS 100, the EDS 300 may comprise the magnet 302 positioned in the housing 304 when incorporated in the medical hardware 400 or the EDS 400 may be provided without the housing 304 and with the magnet 302 freely positioned in the hollow portion 404 of the body 402, within a wall of the body 402, on the outer surface of the body 402, etc. The EDS 300 may be incorporated in the medical hardware using any suitable means, including those described above for the EDS 100. In the illustrated embodiment, the EDS 300 may be positioned toward or proximate a threaded tail end of the body 402. In some embodiments, the EDS 300 may be positioned toward or proximate the a head end of the body 402. The magnet 302 may move relative to the hardware 400 when the EDS 300 is installed. For example, the magnet 302 may move in response to motion of the patient and/or an externally applied magnetic field. Movement of the magnet 302 may produce an oscillating magnetic field.

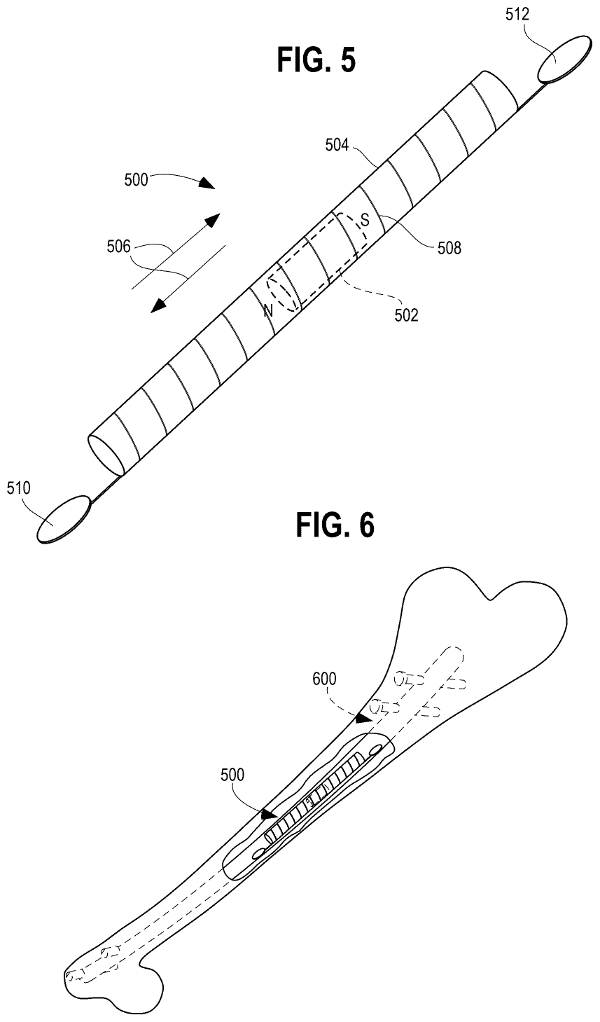

Referring now to FIG. 5, a third example of an energy delivery system (EDS) is indicated at 500. The EDS 500 may include similar components to those of the EDS 100 of FIG. 1 and/or the EDS 300 of FIG. 3. For example, the EDS 500 comprises one or more magnets 502 and a housing 504. The housing 504, like the housing 104, 304 described above, may be a hermetically sealed tube having a cylindrical shape. Description of the housing 104, 304 applies to the housing 504 unless expressly stated otherwise or the context clearly indicates otherwise. The EDS 500 may be used as a standalone device or may be incorporated in medical hardware (e.g., the medical hardware 600 of FIG. 6).

In the illustrated example of FIG. 5, the EDS 500 includes a single magnet 502. In some embodiments, the EDS 500 may include more than one magnet 502, such as two, three, four, five, or more than five magnets. In examples where the EDS 500 includes multiple magnets, the adjacent pairs of the magnets may be positioned with like poles facing one another similar to the arrangement of the magnets 102 of FIG. 1. The magnet(s) 502 may be similar in construction to the magnets 102, 302 described above. For example, the magnet(s) 502 may comprise a permanent magnet. In some embodiments, the magnet(s) 502 may comprise neodymium, ceramic, other suitable magnetic material, or combinations thereof. The size and/or shape of the magnet(s) 502 may vary, e.g., the magnet(s) 502 can have various diameters, sizes, shapes, lengths, etc. depending on the intended application of the EDS 500 and/or size and shape of the housing 504. In the illustrated example, the magnet(s) 502 has a cylindrical shape that complements the cylindrical shape of the housing 504.

The magnet(s) 502 may also be moveable within the housing 504 as described above for the magnets 102, 302. For example, the magnet(s) 502 may freely float or oscillate within the housing 504 in an oscillating direction indicated by the arrows 506 in FIG. 5. Movement of the magnet(s) 502 may be caused by movement of the patient when the EDS 500 is implanted. Additionally or alternatively, the magnet(s) 502 may move in the housing 504 in response to an externally applied magnetic field. Such external magnetic fields may be applied via exogenous magnetic field generators, pulsed or constant magnetic field devices, magnetic patches, or the like. Movement of the magnet(s) 502 in the housing 504 may modulate the position and/or direction of the magnetic field created by the magnet(s) 502. For example, the magnet(s) 502 may move in the housing 504 in the oscillating direction 506 to produce an oscillating magnetic field when the patient is in motion.

The magnet(s) 502 may operate as a primary (or single) magnetic field for the EDS 500. In some embodiments, the magnet(s) 502 may be influenced by an externally applied magnetic field. The magnet(s) 502 may operate as the primary magnetic field influenced by a secondary, externally applied magnetic field, or the magnet(s) 502 may operate as the secondary magnetic field influenced by a primary, externally applied magnet field. Such external magnetic fields may be applied via exogenous magnetic field generators, pulsed or constant magnetic field devices, magnetic patches, or the like.

The EDS 500 also comprises a conductor 508 (e.g., an electrically conductive coil) extending about the housing 504 and/or magnet(s) 502. The conductor 508 may extend about at least a portion of the length of the housing 504 and/or magnet(s) 502 (e.g., extend about less than the full length, the entire length, or greater than the length of the housing 504 and/or magnets 502). The conductor 508 may be made of any conductive material, including, for example and without limitation, copper, aluminum, and combinations thereof. The conductor 508 extends around a path of motion traversed by the magnet(s) 502 when the magnet(s) 502 move in the oscillating direction 506. The conductor 508 may have any shape known in the art (e.g., coil, spiral, helix) that enables the magnet(s) 502 to oscillate through the conductor 508. The conductor 508 may be positioned within the housing 504 or on an outer surface of the housing 504. In the illustrated embodiment, one conductor 508 is provided; in some embodiments, the EDS 500 may comprise more than one conductor, e.g., two conductors, three conductors, etc.

As the magnet(s) 502 oscillate in the housing 504 through the conductor 508, the movement of the magnet(s) 502 creates a change in magnetic flux, generating an electrical current through the conductor 508. In some embodiments, electric current may flow through the conductor 508 and may induce movement of the magnet(s) 502. The conductor 508 may extend between a first electrical contact 510 and a second electrical contact 512. One or both contacts 510, 512 may enable current to flow into and/or from the conductor 508. For example, one or both contacts 510, 512 may receive electrical energy from a generator, an electrical stimulator, or the like. One or both contacts may facilitate the delivery of electric field(s) to the implantation site. The electric field(s) may facilitate the benefits of electric fields at the implantation site as described herein, such as, for example, promoting the healing of the soft tissues, controlling bacterial proliferation and colonization, promoting bone healing, soft tissue-to-bone healing, and soft tissue-to-soft tissue healing, etc. The electric field(s) can be generated via the movement of the magnet(s) 502 through the conductor 508. In some embodiments, the electric field(s) can be induced via an external or exogenous electric field source (e.g., a generator, electrical stimulator). In some embodiments, current flowing through the conductor 508 can influence the magnetic field(s) generated via the magnet(s) 502. The conductor 508 may generate and/or carry AC and/or DC microcurrents and pulse and/or constant currents. In some embodiments, the conductor 508 may generate and/or carry AC current. In some embodiments, the conductor 508 may generate and/or carry AC current that is rectified into DC current. Additional electronic components (e.g., converters) may be included in the EDS 500 to control the generation of electrical energy.

FIG. 6 depicts the EDS 500 incorporated in medical hardware 600. The medical hardware 600 incorporating the EDS 500 may include any medical hardware, including but not limited to orthopedic hardware, bone anchors, dental implants, and the like. In the illustrated example, the medical hardware 600 is an orthopedic nail. For example, the medical hardware 600 may be a femoral nail that can be implanted or installed with the femoral bone or femur of the patient. The medical hardware 600 of FIG. 6 could incorporate the EDS 100 of FIG. 1 and/or the EDS 300 of FIG. 3; the EDS 500 could also be incorporated in the medical hardware 200 of FIG. 2 and/or the medical hardware 400 of FIG. 4. The medical hardware 200, 400, 600 may include similar components (e.g., a hollow or cannulated portion, head, threaded tail, etc.), and description of the medical hardware 200, 400 is applicable to the medical hardware 600, and vice versa, unless expressly stated otherwise or the context clearly indicates otherwise. Additionally, the features of the EDS 100 and/or 300 could be included in the EDS 500, and vice versa, unless expressly stated otherwise or the context clearly indicates otherwise.

The EDS 500 may be incorporated in a structural body of the hardware 600, for example, in a hollow or cannulated portion of the hardware 600, within a wall of the hardware 600, on an outer surface of the hardware 600, or at another suitable location to enable the EDS 500 to function as described herein. The EDS 500 may comprise the magnet(s) 502, the conductor 508, and the housing 504 when incorporated in the medical hardware 600, or the EDS 500 may be provided without the housing 504 such that the magnet(s) 502 and the conductor 508 are freely positioned in the hollow portion of the hardware 600, within a wall of the hardware 600, on the outer surface of the hardware 600, etc. The EDS 500 may be incorporated in the medical hardware 600 using any suitable means, including those described above for the EDS 100, 300. The magnet(s) 502 may move relative to the hardware 600 when the EDS 500 is installed. For example, the magnet(s) 502 may move in response to motion of the patient and/or an externally applied magnetic field and/or current flowing through the conductor 508. Movement of the magnet(s) 502 may produce an oscillating magnetic field. Current flowing through the conductor 508, which may be generated via oscillation of the magnet(s) 502 and/or via an external electrical energy source, may comprise AC and/or DC microcurrents and pulse and/or constant currents.

In some embodiments, the componentry of the EDS 100, 300, 500 (e.g., the magnets 102, 302, 502 and/or the conductor 508) may cooperate with the structure of the housing 104, 304, 504 and/or the medical hardware 200, 400, 600 to enhance functionality in conjunction with the delivery of the magnetic and/or electric fields to the target area. Various modifications could be made to the structure of the EDS housing 104, 304, 504 and/or the medical hardware 200, 400, 600 for this purpose. For example, the housing 104, 304, 504 and/or the medical hardware 200, 400, 600 may be selectively coated with, or otherwise selectively incorporate, electrically insulating material or magnetic shielding material to control direction and/or intensity of the magnetic and/or electric fields. In some embodiments, the housing 104, 304, 504 and/or the medical hardware 200, 400, 600 may include perforations or openings in an outer surface there that utilize the indwelling magnetic field to facilitate bone-to-housing or bone-to-hardware ingrowth, which may facilitate accelerated and improved fixation between bone and the housing 104, 304, 504 and/or the medical hardware 200, 400, 600.

### Example Treatment Methods

Also provided herein are methods of treating an implantation site of a medical hardware device implanted in a patient via magnetic and/or electric field therapy. The methods comprise generating magnetic and/or electric fields utilizing any embodiment of the EDS described herein (e.g., the EDS 100, the EDS 300, and/or the EDS 500) when installed at an implantation site of the patient. The EDS may be equipped with the componentry, features, and functionality described herein for generating the magnetic and/or electric fields (e.g., free floating or oscillating magnet(s), an electrical conductor for producing current via electromagnetic interaction with the magnet(s), etc.). In some embodiments, the generating of the magnetic and/or electric fields comprises generating a constant or pulsed magnetic and/or electric field. In some embodiments, the generating of the magnetic and/or electric fields comprises receiving, via the EDS, magnetic and/or electric energy from an external energy source (e.g., a generator, electrical stimulator, pulsed or constant magnetic field device, external magnet, and the like). In some embodiments, the generating of the magnetic and/or electric fields comprises producing one or more magnetic field gradients using the EDS and modulating the magnetic field gradients. In some embodiments, the generating of the magnetic and/or electric fields comprises generating a flow of AC and/or DC current in a conductor of the EDS using electromagnetic interaction between the conductor and one or more permanent magnets oscillating relative to the conductor.

In some embodiments, the EDS generates the magnetic and/or electric fields when installed at a subfascial location. In some embodiments, the EDS generates the magnetic and/or electric fields when installed in bone or bone tissue. The EDS may be utilized as a standalone device when generating magnetic and/or electric fields or may be incorporated in medical hardware (e.g., the hardware 200, the hardware 400, and/or the hardware 600). For example, the EDS may be incorporated in any medical hardware, including but not limited to orthopedic hardware, bone anchors, dental implants, and the like, and can generate magnetic and/or electric fields at the implantation site where such hardware is installed. The methods also comprise delivering the magnetic and/or electric fields to a target area proximate the implantation site. The magnetic and/or electric fields may facilitate the benefits described herein, for example, promoting the healing of the soft tissues, controlling bacterial proliferation and colonization, promoting bone healing, soft tissue-to-bone healing, and soft tissue-to-soft tissue healing, reducing risk of infection, etc.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, references to “one embodiment” or “an embodiment” of the present disclosure are not intended to be interpreted as excluding the existence of additional embodiments that also incorporate the recited features.

All documents described herein are incorporated by reference herein, including any priority documents and or testing procedures to the extent they are not inconsistent with this text.

The specific embodiments described herein have been illustrated by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function) . . . ” or “step for (perform)ing (a function) . . . ”, it is intended that such elements are to be interpreted under 35 U.S.C. 112 (f). However, for any claims containing elements designated in any other manner, it is intended that such elements are not to be interpreted under 35 U.S.C. 112 (f).

While the present disclosure has been described with respect to a number of embodiments and examples, those skilled in the art, having benefit of this disclosure, will appreciate that other embodiments can be devised which do not depart from the scope and spirit of the present disclosure.

What is claimed is:

1. A medical hardware device comprising:
a structural body implantable at an implantation site of a patient; and
an energy delivery system (EDS) incorporated in the structural body, the EDS comprising componentry operable to generate, directly or passively, magnetic and/or electric fields, wherein the EDS is configured to deliver the magnetic and/or electric fields to a target area proximate the implantation site.

2. The medical hardware device of claim 1, wherein the componentry of the EDS comprises one or more permanent magnets.

3. The medical hardware device of claim 2, wherein the one or more permanent magnets are moveable relative to the structural body.

4. The medical hardware device of claim 3, wherein the one or more permanent magnets freely float relative to the structural body such that the one or more permanent magnets are moveable in response to motion of the patient.

5. The medical hardware device of claim 3, wherein the one or more permanent magnets are moveable in response to an externally applied magnetic field.

6. The medical hardware device of claim 3, wherein the componentry of the EDS further comprises an electrical conductor extending around a path of motion of the one or more permanent magnets, wherein movement of the one or more permanent magnets causes and/or is caused by current flowing through the electrical conductor.

7. The medical hardware device of claim 6, wherein the electrical conductor is electrically connected to at least one electrical contact receiving the current flowing through the electrical conductor.

8. The medical hardware device of claim 2, wherein the one or more permanent magnets comprise neodymium or ceramic material.

9. The medical hardware device of claim 1, wherein the componentry of the EDS is positioned in a hermetically sealed housing.

10. The medical hardware device of claim 9, wherein the housing is sized and shaped for positioning in a hollow portion of the structural body.

11. The medical hardware device of claim 1, wherein the componentry of the EDS comprises at least two permanent magnets, wherein each adjacent pair of the at least two permanent magnets has like poles facing one another and creates a magnetic field gradient.

12. The medical hardware device of claim 11, wherein the at least two permanent magnets are moveable relative to one another and to the structural body, wherein movement of the at least two permanent magnets modulates the magnetic field gradient.

13. The medical hardware device of claim 1, comprising orthopedic hardware, a bone anchor, or a dental implant.

14. The medical hardware device of claim 1, comprising a nail, a screw, a plate, an anchor, a peg, a pin, a rod, a fixator, a ring, or a bolt.

15. The medical hardware device of claim 1, wherein the magnetic and/or electric fields facilitate tissue healing, osseointegration, pain relief, and/or infection mitigation in the target area.

16. A method of treating an implantation site of a medical hardware device implanted in a patient, the method comprising:
generating magnetic and/or electric fields using an energy delivery system (EDS) positioned in a subfascial, bone, or tissue region proximate the implantation site; and
delivering the magnetic and/or electric fields to a target area proximate the implantation site.

17. The method of claim 16, wherein the generating of the magnetic and/or electric fields comprises generating a constant or pulsed magnetic and/or electric field.

18. The method of claim 16, wherein the generating of the magnetic and/or electric fields comprises receiving, via the EDS, magnetic and/or electric energy from an external energy source.

19. The method of claim 16, wherein the generating of the magnetic and/or electric fields comprises producing one or more magnetic field gradients using the EDS and modulating the magnetic field gradients.

20. The method of claim 16, wherein the generating of the magnetic and/or electric fields comprises generating a flow of AC and/or DC current in a conductor of the EDS using electromagnetic interaction between the conductor and one or more permanent magnets oscillating relative to the conductor.
